(19) United States
(12) Patent Application Publication
(10) Pub. No.: US 2010/0286284 A1
(43) Pub. Date: Nov. 11, 2010

(54) 5-[1'-(DECAHYDRO-7-HYDROXY-1,1,3A,7-TETRAMETHYL-1H-CYCLOPROPA[A]NAPHTHALEN-4-YL)-3'-METHYLBUTYL]-2,4,6-TRIHYDROXY-1,3-BENZENEDICARBOXALDEHYDE AS MEDICAMENTS

(75) Inventors: Christel Fiorini-Puybaret, Toulouse (FR); Philippe Joulia, Vilenouvelle (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Pierre Fabre Medicament, Boulogne Billancourt (FR)

(21) Appl. No.: 12/735,457

(22) PCT Filed: Jan. 13, 2009
(86) PCT No.: PCT/FR2009/000038

§ 371 Date: Jul. 16, 2010

(30) Foreign Application Priority Data
Jan. 18, 2008 (FR) 0800276

(51) Int. Cl. A61K 31/11; C07C 47/57; A61P 25/30; A61P 25/22; A61P 25/18; A61P 25/06; A61P 25/14; A61P 3/04

(52) U.S. Cl. 514/700; 568/441

(57) ABSTRACT

The present invention relates to new compounds of formula (I) and to their use as medicaments. Preferably, the said compounds are used in the preparation of a medicament or food supplement intended for the treatment and/or prevention of ailments or pathologies ensuing from a disorder of the reuptake of the following neurotransmitters: dopamine, serotonin and/or noradrenaline.

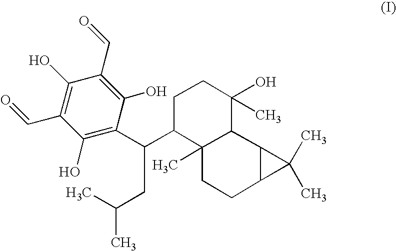

The present invention relates to new compounds of formula (I), which belong to the macrocarpal family, and to their use as medicaments. Preferably, said compounds will be used in the preparation of a medicament or food supplement intended for the treatment and/or prevention of ailments or pathologies ensuing from a disorder of the reuptake of the following neurotransmitters: dopamine, serotonin and/or noradrenaline.

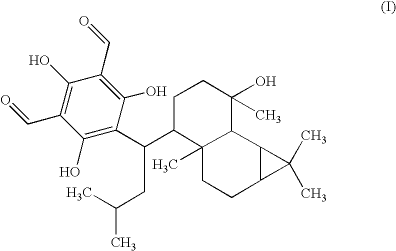

Macrocarpals are compounds of the acyl-phloroglucinol chemical family. In the plant kingdom, they are present principally in various species of the Eucalyptus genus. Cosmetic properties have been described for, inter alia, macrocarpals L and M (JP 2001055325 and SHIBUYA Y. et al., 2001—Isolation and structure determination of new Macrocarpals from a Herbal Medecine, Eucalyptus globulus Leaf—Natural Medecines 55 (1), 28-31).

Whilst conducting active research into neurotransmitter reuptake inhibitors, the Applicant has found, in especially surprising manner, that new compounds of the macrocarpal family and, more especially, macrocarpal L and M have pharmacological properties in this field.

One of the objectives of the present invention is to provide new chemical compounds of formula (I) for which therapeutic applications can be envisaged.

The present invention accordingly relates also to said compounds of formula (I) as medicaments and also to pharmaceutical compositions or food supplements comprising at least one compound of formula (I) as active ingredient.

Finally, another objective of the present invention is use of said compounds (I) in the treatment and/or prevention of neurological or psychiatric pathologies or ailments and associated disorders, functional somatic disorders, obesity, overweight and dependence on addictive substances, ensuing from a disorder of the reuptake of dopamine and/or serotonin and/or noradrenaline.

The compounds according to the present invention correspond to the following general formula (I):

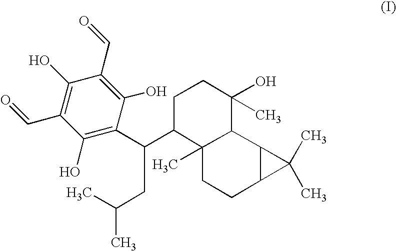

Empirical formula: C₂₈H₄₀O₆

Molecular weight: 472 g/mol

In the context of the present invention, “compounds of formula (I)” are understood to mean: the totality of the enantiomeric and diastereoisomeric forms of 5-[1′-(decahydro-7-hydroxy-1,1,3a,7-tetramethyl-1H-cyclopropa[a]naphthalen-4-yl)-3′-methylbutyl]-2,4,6-trihydroxy-1,3-benzenedicarboxaldehyde, as well as mixtures thereof.

Hereinbelow, the Applicant specifies the numbering used for the carbon atoms:

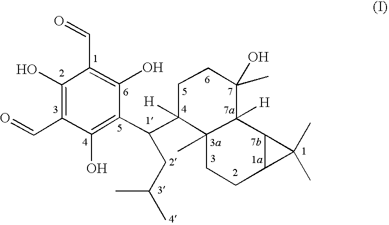

The formula (I) comprises 7 asymmetric carbon atoms designated by <<C*>> in the formula hereinbelow:

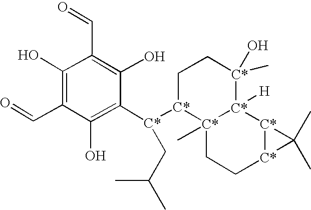

The present invention includes new compounds corresponding to formula (I) with the exception of the following two forms:
5-[(1′R)-1′-[(1aS,3aS,4S,7R,7aR,7bS)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde; and
5-[(1′S)-1′-[(1aS,3aS,4S,7R,7aR,7bS)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde.

A further objective of the present invention is directed to compounds of formula (I) as defined on the preceding page for which a first therapeutic application has been established by the Applicant.

Those compounds of formula (I) used alone or in admixture as medicaments are thus also described in the present invention.

Preferably, said compounds (I) as medicaments according to the invention are selected from the group composed of:
5-[(1′R)-1′-[(1aS,3aS,4S,7R,7aR,7bS)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde; of the formula below:

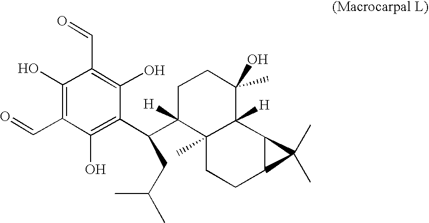

5-[(1′S)-1′-[(1aS,3aS,4S,7R,7aR,7bS)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde; of the formula below:

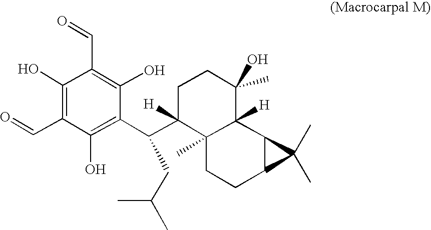

5-[(1′R)-1′-[(1aR,3aS,4S,7R,7aR,7bR)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde; and
5-[(1′S)-1′-[(1aR,3aS,4S,7R,7aR,7bR)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde;
   used alone or in admixture.

In a particular embodiment of the present invention, the compounds of formula (I) are obtained from a plant extract. Advantageously, the plant extract originates from a Eucalyptus.

In the context of the present invention, <<Eucalyptus>> is understood to mean species belonging preferably to the sub-genera Eudesmia, Symphomyrtus and Corymbia, and more especially the following species: Eucalyptus globulus L., Eucalyptus pulverulenta Sims, Eucalyptus kartzoffiana L. A. S. Johnson 1 Blaxell, Eucalyptus macrocarpa Hook., Eucalyptus cinerea F. Muell.ex Benth., Eucalyptus dorrigoensis (Blakely) L. A. S. Johnson 1 K. D. Hill, Eucalyptus leptopoda Benth., Eucalyptus occidentalis Endl., Eucalyptus viridis R. T. Baker, Eucalyptus polybractea R. T. Baker and Eucalyptus smithii R. T. Baker.

These examples illustrate the present invention without, however, the scope thereof being limited thereto.

The Eucalyptus extract is advantageously obtained from leaves, blossom, fruit, seeds, stems or trunk of Eucalyptus; and preferably from Eucalyptus leaves.

The present invention relates also to pharmaceutical compositions or food supplements comprising at least one compound of formula (I) as active ingredient. Preferably, the compounds of formula (I) are incorporated in the form of plant extracts containing them.

In a particular embodiment of the present invention, the proportion by weight of compound (I) according to the invention, and preferably of macrocarpal L or macrocarpal M, in a Eucalyptus extract is higher than 0.05% and strictly lower than 90%.

Said plant extract, including Eucalyptus extract, can be obtained by an extraction process carried out by conventional steps known to the person skilled in the art.

The leaves, blossom, fruit, seeds, stems or trunk of Eucalyptus (Eucalyptus sp.) or a mixture of those parts are crushed and then extracted with an organic solvent which may be an alkane (pentane, hexane, heptane, octane, cyclohexane), an ether (tetrahydrofuran, dioxane, diethyl ether), an ester (ethyl acetate, isopropyl acetate), an alcohol (methanol, ethanol, propanol, isopropanol, butanol, octanol), a ketone (methyl ethyl ketone, methyl isobutyl ketone), a halogenated hydrocarbon (chloroform, dichloromethane) or a mixture of water and water-miscible organic solvent(s) (a water-alcohol mixture, for example). The extraction is carried out with a plant/solvent ratio of from approximately 1/1 to approximately 1/20 and may be repeated 2 to 3 times. The temperature of the extraction solvent may be ambient temperature or higher, and may be up to the boiling temperature of the solvent employed. The contact time of the plant with the solvent is from approximately 30 min. to approximately 72 hours.

A solid-liquid separation is then carried out, the plant being separated from the solvent by filtration or centrifugation.

The filtrate obtained may either:
be concentrated to dryness directly by complete evaporation of the extraction solvent, and constitute the final extract;
or be concentrated to a greater or lesser extent. In the case of a mixed extraction solvent (water/alcohol mixture, for example), concentration is continued until the organic solvent present has been evaporated off. In the case of an organic solvent, a quantity of water is added to the concentrate obtained. A liquid-liquid purification step is carried out by adding to the aqueous phase an immiscible solvent, which may be an alkane (hexane, for example), an ether (diethyl ether, for example), an ester (ethyl acetate, for example), an alcohol (butanol, for example), a ketone (methyl ethyl ketone, for example) or a halogenated hydrocarbon (chloroform, for example). One, two or three liquid-liquid extractions are carried out. The combined organic phases may be dried over sodium sulfate before being concentrated to dryness.

The solutions obtained are concentrated in vacuo and at a temperature ranging from ambient temperature to the boiling temperature.

The final extract is dried by lyophilisation or by more conventional means of drying known to the person skilled in the art (spray-drying, oven . . . ).

The drying temperatures preferably do not exceed about 60° C.

The extract can be stabilised by adding an antioxidant, such as, for example, ascorbic acid or citric acid in amounts ranging from approximately 0.05 to approximately 1 g per 100 g of dry extract.

The extract so obtained comprises a proportion by weight of compound of formula (I) that is higher than 0.05% and strictly lower than 5%, said proportion by weight preferably being about 0.8%.

The extract obtained above may be rich in compounds of formula (I) and more especially in macrocarpal L or macrocarpal M.

In the context of the present invention, <<Eucalyptus extract rich in macrocarpal L or M>>, is understood to mean a Eucalyptus extract in which the proportion by weight of macrocarpal L or M is higher than or equal to 5% and strictly lower than 90%, preferably higher than or equal to 5% and lower than 50%, more preferably higher than or equal to 5% and lower than 30% and even more preferably higher than or equal to 5% and lower than 15%.

The process for obtaining said extract comprises the following steps:
crushing leaves and/or blossom and/or fruit and/or seeds and/or stems and/or trunk of Eucalyptus;
at least one extraction with an organic solvent or a mixture of water and water-miscible organic solvent(s).
The extraction is carried out with a plant/solvent ratio of from approximately 1/1 to approximately 1/20 and may be repeated 2 to 3 times. The temperature of the extraction solvent may be ambient temperature or higher, and may be up to the boiling temperature of the solvent employed. The contact time of the plant with the solvent is from approximately 30 min. to approximately 72 hours.
Preferably, the solvent is selected from the group composed of an alkane (pentane, hexane, heptane, octane, cyclohexane), an ether (tetrahydrofuran, dioxane, diethyl ether), an ester (ethyl acetate, isopropyl acetate), an alcohol (methanol, ethanol, propanol, isopropanol, butanol, octanol), a ketone (methyl ethyl ketone, methyl isobutyl ketone), a halogenated hydrocarbon (chloroform, dichloromethane) or a mixture of water and water-miscible organic solvents (a water-alcohol mixture, for example).
Preferably, the extraction solvent is dichloromethane or isopropyl acetate.
In the case of a water-miscible extraction solvent, the filtrate is concentrated to dryness then dissolved in a water-immiscible solvent.
In the case of a water-immiscible solvent, the filtrate is concentrated.
Liquid/liquid separation by techniques known to the person skilled in the art.

In a preferred embodiment of the invention, one or more liquid-liquid extractions are carried out by addition of a base, preferably sodium carbonate (Na₂CO₃). The combined basic aqueous phases are acidified by the addition of acid, preferably hydrochloric acid (HCl), then extracted by one or more liquid-liquid extractions carried out with a water-immiscible solvent. Advantageously, the acidification results in a pH of approximately 1.

The combined organic phases may be dried over sodium sulfate and then concentrated in vacuo at a temperature ranging from ambient temperature to boiling temperature. The concentrate is dried by conventional drying means (spray-drying, oven . . . ) at temperatures preferably not exceeding 60° C. and constitutes the extract rich in macrocarpal L or macrocarpal M. The extract can be stabilised by addition of an antioxidant such as, for example, ascorbic acid or citric acid in amounts of from 0.05 to 1 g per 100 g of dry extract.

In a particular embodiment of the invention, the extraction solvent may be a supercritical fluid.

Leaves, blossom, fruit, seeds, stems or trunk of Eucalyptus (Eucalyptus sp.) or a mixture of those parts may or may not be crushed, and are then extracted with a supercritical fluid which may be carbon dioxide.

A first extraction, preferably with supercritical CO₂, is carried out under the following conditions:
the temperature of the fluid is from approximately 40° C. to approximately 80° C., and preferably from approximately 40° C. to approximately 60° C.;
its pressure is from approximately 80 bars to approximately 250 bars, and
preferably from approximately 100 bars to approximately 200 bars;
the duration of the extraction is from approximately 1 hour to approximately 6 hours;
the flow rate of the fluid will be adapted by the person skilled in the art as a function of the amount of material to be extracted and the size of the autoclave used. Preferably, the flow rate of CO₂ applied in the process according to the invention is from 2 to 15 kg/hour, advantageously from 8 to 12 kg/hour;
   for an amount of plant of from 200 to 1000 grams, preferably of approximately 500 grams,
   and for an autoclave having a capacity of from 2 to 10 litres, preferably a capacity of approximately 5 litres.

During that first extraction step, it is possible to add an organic co-solvent from the family of the alcohols (including ethanol), ethers or esters, or a mixture of two or more of those solvents.

The plant so extracted may then optionally be subjected to a second extraction. The extraction fluid is preferably supercritical CO₂ with or without co-solvent. The operating conditions are as follows:
the temperature of the fluid is from approximately 40° C. to approximately 80° C., and preferably from approximately 40° C. to approximately 60° C.;
its pressure is from approximately 80 bars to approximately 250 bars, and preferably from approximately 100 bars to approximately 200 bars;
the flow rate of the fluid is from 2 to 15 kg/hour, advantageously from 8 to 12 kg/hour;
   for an amount of plant of from 200 to 1000 grams, preferably of approximately 500 grams,
   and for an autoclave having a capacity of from 2 to 10 litres, preferably a capacity of approximately 5 litres.

Advantageously, the extraction is carried out in a plant/co-solvent weight ratio of approximately from 1/0.1 to 1/5.

This second extraction step can be repeated if necessary. The duration of extraction is from approximately 1 hour to approximately 3 hours per additional extraction step.

The extract obtained is then subjected to evaporation.

The person skilled in the art will adjust the operating conditions of the procedure by means of supercritical fluid to obtain a Eucalyptus extract that is enriched to a greater or lesser extent.

The final extract is dried by lyophilisation or by more conventional means of drying known to the person skilled in the art (spray-drying, oven, . . . ). The drying temperatures preferably do not exceed about 60° C.

The extract can be stabilised by adding an antioxidant, such as, for example, ascorbic acid or citric acid in amounts ranging from approximately 0.05 to approximately 1 g per 100 g of dry extract.

In a preferred manner, said compounds (I), including macrocarpal L and macrocarpal M, may be isolated from a plant extract. The plant extract is preferably a Eucalyptus extract. Techniques that allow them to be purified are chromatographic techniques conventional for the person skilled in the art. The extracts are fractionated on a preparative column which has as stationary phase an inverse phase, preferably Symetry Shield®, 5 μm (Waters), and as mobile phase an acetonitrile/water/trifluoroacetic acid mixture in the proportions 95/5/0.1%.

The purity in compound of formula (I) of such a fraction is greater than or equal to 90%. Preferably, the purity in macrocarpal L or macrocarpal M of such a fraction is greater than or equal to 90%.

The Applicant has demonstrated the influence of the compounds of formula (I) according to the invention on the reuptake of neurotransmitters.

In the context of the present invention, <<neurotransmitters>> are understood to mean: dopamine and/or serotonin and/or noradrenaline.

In view of their pharmacological properties of inhibiting the reuptake of those neurotransmitters, said compounds of formula (I) are especially useful in the preparation of a medicament or food supplement intended for the treatment and/or prevention of numerous ailments or pathologies resulting from a lack of dopamine and/or serotonin and/or noradrenaline.

Among the ailments or pathologies that can be treated and/or prevented by using at least one compound of formula (I) according to the present invention, there may be mentioned as non-limiting illustrative examples:
neurological diseases, ailments or disorders:
   such as neurodegenerative diseases (Alzheimer's disease, Huntington's chorea, Parkinson's disease, cerebral vascular accidents, cranial traumatism), amyotrophic lateral sclerosis, senile dementias, fronto-temporal dementias, vascular dementias, migraine, neuropathic pains of central origin;
psychiatric diseases, ailments or disorders:
   such as depression (endogenous, resistant, reactive or iatrogenic), depressive state, schizophrenia, bipolar disorder, generalised anxiety, diseases associated with stress, panic attacks, obsessive compulsive disorders, post-traumatic stress syndromes, attention and hyperactivity disorders, eating behaviour disorders (especially bulimia, anorexia), phobia (especially agrophobia), autism;
memory, attention and vigilance disorders associated with neurological and psychiatric diseases, ailments and disorders;
functional somatic disorders:
   such as chronic fatigue syndrome, fibromyalgia, irritable bowel syndrome, gastro-oesophageal refluxes, loss of libido, erection disorders, urinary incontinence.
obesity, overweight
dependence on addictive substances:
   especially nicotine, alcohol, opiates; cannabinoids, psychostimulants. Indeed the medicament or food supplement according to the invention is advantageously intended:
to treat dependence on addictive substances; and more especially during the stage prior to stopping smoking by contributing, inter alia, to a reduction in consumption and/or a reduction in associated symptoms, such as anxiety states and/or depressive states.
to reduce dependence on nicotine, alcohol, opiates, cannabinoids, psychostimulants; and thus allow consumption to be ceased.
to prevent relapse in abstinent individuals and to maintain abstinence;
to prevent and/or reduce the symptoms associated withdrawal from nicotine, alcohol, opiates, cannabinoids or psychostimulants, such as anxiety states and/or depressive states.

The person skilled in the art will be able to recognize other pathologies where treatment requires such inhibition. The Applicant quotes herein, without implying any limitation, a number of bibliographic references that review the link between pathologies and their treatment with a triple reuptake inhibitor of dopamine and/or serotonin and/or noradrenaline. An example of each <<group>> has been given.

Dopamine, serotonin and noradrenaline co-operate in the development and continued existence of neurons (Lauder J. M., Trends Neurosci, 1993, 16; 233). Certain neurological pathologies, such as Parkinson's disease (Hornykiewicz O., Adv Cytopharmacol. 1971, 1; 369) result from a deficiency in dopamine; monoamine oxydase inhibitors, which increase the levels of dopamine, serotonin and noradrenaline, are used to treat Parkinson's disease and other neurological ailments (Ebadi M., Curr Drug Targets. 2006, 7; 1513). The compounds of formula (I) according to the present invention can thus very advantageously be used in the treatment of such neurological diseases.

Depression is a common mood pathology, which is characterised by feelings of intense sadness, pessimistic thoughts and self-depreciation, often accompanied by loss of drive, of enthusiasm and of libido. The inability to feel pleasure from normally pleasant experiences, also known by the name of anhedonia, is also considered to be a common symptom of depression. Depression is currently treated by selective serotonin reuptake inhibitors, such as fluoxetine, citalopram or paroxetine, selective noradrenaline reuptake inhibitors, such as reboxetine, or also mixed serotonin and noradrenaline reuptake inhibitors, such as milnacipran or venlafaxine. However, a significant role in pleasure and motivation has been attributed to dopaminergic neurons projecting to a region of the brain called the nucleus accumbens, (Koob G. F. Sem. Neurosci. 1992, 4, 139; Salamone J. D. Behay. Brain Res. 1994, 61, 117). Symptoms of depression can thus very advantageously be treated by a dopamine, serotonin and noradrenaline reuptake inhibitor, such as a compound of formula (I) according to the present invention.

The absorption of addictive substances, including nicotine, increases the extracellular levels of dopamine in the ventral striatum of animals (Di Chiara G and Imperato A., Proc Natl Acad Sci USA. 1988, 85; 5274) and man (Brody et al., Am J Psychiatry, 2004, 161; 1211). Nicotine withdrawal may be accompanied by a depressive syndrome (Wilhelm K et al., Drug Alcohol Rev, 2006, 25; 97). The compounds of formula (I) according to the present invention can thus advantageously be used as a substitution treatment for addictive substances, such as nicotine, and for the prevention or treatment of the depressive syndrome associated withdrawal.

Functional disorders, also called somatotropic disorders, are disorders concerned with the major physiological functions, and which would be due not to organic lesions but to the manner in which the organs (liver, heart . . . ) function. Functional somatic disorders may occur at the start of a disease that will manifest itself later. Among those disorders, fibromyalgia is a disorder combining diffuse or localised pain, chronic fatigue, depressive symptoms, memory disorders and concentration disorders (Rooks D S., Curr Opin Rheumatol. 2007, 19; 111). Fibromyalgia symptoms are treated with mixed noradrenaline/serotonin reuptake inhibitors (Vitton O., Hum Psychopharmacol. 2004, 19 Suppl 1:S27). The addition of a component enhancing dopaminergic tonus, such as a compound of formula (I) according to the present invention, is advantageous in the preparation of a medicament or food supplement intended for the treatment and/or prevention of functional somatic disorders.

Advantageously, said medicament is presented in an oral, injectable or transdermal form.

The oral form is advantageously selected from the group composed of tablets, hard capsules, soft capsules, liquid preparations such as syrups, drinkable solutions and powders for drinkable suspensions.

The said food (or nutraceutical or dietetic) supplement is advantageously packaged in the form of doses, that is in presentation forms such as capsules, pastilles, tablets, pills and other similar forms, and also powder sachets, liquid ampoules, bottles fitted with drop dispensers, and other analogous forms of liquid and powder preparations that are to be taken in small-quantity measured units.

The invention will be better understood with the aid of the following Examples, which do not, however, limit the scope of the invention.

### EXAMPLE 1

### Preparation of Macrocarpal L from a Eucalyptus globulus Extract

Eucalyptus globulus leaves are crushed and then extracted with 5 volumes of dichloromethane. The extraction is carried out twice, at reflux, for 1 hour.

Filtration in vacuo is then carried out. The combined filtrates are concentrated to 2 volumes.

Three liquid-liquid extractions are carried out by addition of one volume of 0.1M sodium carbonate (Na₂CO₃).

The combined basic aqueous phases are acidified by addition of 1M hydrochloric acid (HCl) until a pH of about 1 is obtained, then extracted by 3 liquid-liquid extractions with dichloromethane. The organic phases are dried over sodium sulphate, then concentrated, and concentrated to dryness in vacuo at 60° C. maximum. The dry residue obtained contains a proportion by weight of macrocarpal L of 1.5%. The extract so obtained is fractionated on a silica column with a discontinuous gradient of toluene/acetone varying in the following proportions: toluene 100%, toluene/acetone: 99/1, acetone 100%. The 99/1 toluene/acetone fraction containing macrocarpal L is evaporated, dried and then purified on a preparative column having as stationary phase an inverse phase, Symetry Shield®, 5 μm (Waters) and as mobile phase an acetonitrile/water/trifluoroacetic acid mixture in the proportions 25/75/0.1%.

The purity in macrocarpal L of the fraction obtained is approximately 97%.

The NMR data of the isolated molecule are as follows:

¹H NMR (500 MHz, PYRIDINE-d5) δ ppm 0.62 (t, J=8.54 Hz, 1H) 0.91 (dd, J=9.16, 5.80 Hz, 1H) 0.97 (d, J=6.41 Hz, 3H) 0.99-1.05 (m, 1H) 1.06 (d, J=6.10 Hz, 3H) 1.10 (s, 3H) 1.11 (s, 3H) 1.24 (s, 3H) 1.42-1.46 (m, 1H) 1.44 (s, 3H) 1.50-1.65 (m, 3H) 1.66-1.77 (m, 2H) 1.90 (td, J=13.12, 3.36 Hz, 1H) 1.92-2.02 (m, 1H) 2.07 (dt, J=12.28, 3.01 Hz, 1H) 2.13-2.24 (m, 2H) 2.74 (td, J=12.21, 3.05 Hz, 1H) 3.86 (dt, J=11.37, 4.54 Hz, 1H) 10.56 (s, 1H) 10.57 (s, 1H) ¹³C NMR (125 MHz, PYRIDINE-d5) δ ppm 16.3, 16.5, 17.1, 17.8, 19.2, 22.3, 22.4, 24.4, 25.0, 25.7, 27.8, 30.1, 32.3, 39.0, 39.3, 44.9, 45.4, 52.3, 54.0, 72.2, 107.0, 107.6, 107.8, 171.6, 172.1, 173.0, 192.2, 192.4

### EXAMPLE 2

### Determination of the 50% Inhibitory Concentration (IC₅₀) of Macrocarpal L Prepared According to Example 1 with Respect to the Reuptake of Neurotransmitters Compared with that of Hyperforin

The uptake tests were carried out in vitro on rat synapses.

### 1) Evaluation of the Reuptake of Serotonin (or 5-HT)

The protocol used for this evaluation is that described in Perovic, S. and Muller W. E. G., 1995—Pharmacological profile of hypericum extract: effect on serotonin uptake by postsynaptic receptors, Arzneim-Forsch. Drug Res., 45: 1145-1148.

The principle of it is as follows:

Synapses from rats' brains are incubated for 15 min at 37° C. with 0.1 μCi [³H]-serotonin in the presence or absence (control) of the compound prepared according to Example 1 or of imipramine (reference) in a buffer containing 118 mM NaCl, 5 mM KCl, 2.5 mM MgSO₄, 1.2 mM NaH₂PO₄, 25 mM NaHCO₃, 11 mM glucose, 10 μM EGTA and 50 μM ascorbic acid (pH=7.4).

The basal activity is determined by incubating the same mixture for 15 min at 37° C. in the presence of 10 μM imipramine to block the reuptake.

Following incubation, the samples are rapidly filtered in vacuo through glass fibre filters (GB/B, Packard) and rinsed twice with ice-cold incubation buffer to eliminate free [³H]-serotonin. The filters are dried and the radioactivity retained is measured by a scintillation counter (Topcount, Packard) using a scintillation cocktail (Microscint O, Packard).

### 2) Evaluation of the Reuptake of Dopamine (or DA)

The protocol used for this evaluation is that described in Janowsky A. Berger P., Vocci F., Labarca R., Skolnick P., and Paul S. M., 1996—Characterization of sodium-dependent [³H]GBR-12935 binding in brain: a radioligand for selective labelling of the dopamine transport complex, J. Neurochem., 46, 1272-1276.

The principle of it is as follows:

The synaptic medium (synapses of rat striatum) is incubated for 15 min at 37° C. with 0.1 μCi [³H]-DA in the presence or absence (control) of the compound prepared according to Example 1 or of GBR 12909 (reference) in the buffer solution (cf. reuptake of serotonin).

The basal activity is determined by incubating the same mixture for 15 min at 37° C. in the presence of 10 μM of GBR 12909 to block the reuptake.

Following incubation, the samples are rapidly filtered in vacuo through glass fibre filters (GB/B, Packard) and rinsed twice with ice-cold incubation buffer to eliminate free [³H]-dopamine. The filters are dried and the radioactivity retained is measured by a scintillation counter (Topcount, Packard) using a scintillation cocktail (Microscint O, Packard).

### 3) Evaluation of the Reuptake of Noradrenaline (or NE)

The protocol used for this evaluation is that described in Perovic, S. and Muller W. E. G., 1995—Pharmacological profile of hypericum extract: effect on serotonin uptake by postsynaptic receptors, Arzneim-Forsch. Drug Res., 45: 1145-1148.

The principle of it is as follows:

The synaptic medium (synapses of rat hypothalamus) is incubated for 20 min at 37° C. with 0.1 μCi [³H]-NE in the presence or absence (control) of the compound prepared according to Example 1 or of protriptyline (reference) in the buffer solution (cf. reuptake of serotonin).

The basal activity is determined by incubating the same mixture for 20 min at 37° C. in the presence of 10 μM of protriptyline to block the reuptake.

Following incubation, the samples are rapidly filtered in vacuo through glass fibre filters (GB/B, Packard) and rinsed twice with ice-cold incubation buffer to eliminate free [³H]-NE. The filters are dried and the radioactivity retained is measured by a scintillation counter (Topcount, Packard) using a scintillation cocktail (Microscint O, Packard).

### 4) Results:

The results are expressed as a percentage inhibition of the reuptake of the neurotransmitter evaluated.

Those different protocols were repeated for different concentrations of the compound prepared according to Example 1 and of hyperforin.

The inhibition curves obtained allowed the following IC₅₀ values to be obtained:

TABLE 1
Determination of the 50% inhibitory concentration (IC₅₀) of the
compound according to the present invention and of hyperforin with
respect to the reuptake of serotonin, noradrenaline and dopamine

IC₅₀ (μg/ml of solution)

| Test | Macrocarpal L | Hyperforin |
|---|---|---|
| Serotonin reuptake | 1.8 | 0.89 |
| Noradrenaline reuptake | 3.1 | 0.79 |
| Dopamine reuptake | 1.0 | 0.23 |

These results show that the compound of formula (I) according to the present invention has an inhibitory activity on the reuptake of neurotransmitters.

1-12. (canceled)

13. A compound of formula (I) 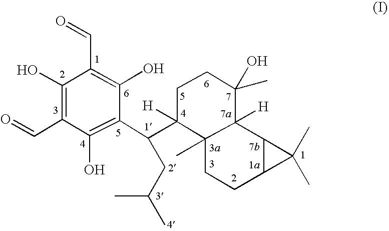
wherein the following forms are excluded:
5-[(1′R)-1′-[(1aS,3aS,4S,7R,7aR,7bS)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde; and
5-[(1′S)-1′-[(1aS,3aS,4S,7R,7aR,7bS)-decahydro-7-hydroxy-1,1,3a,7-tetramethyl-1H-cyclopropa[a]naphthalen-4-yl]-3′-methylbutyl]-2,4,6-trihydroxy-1,3-benzenedicarboxaldehyde.

14. A compound of claim 13, which is obtained from a plant extract.

15. A compound of claim 14, wherein the plant extract originates from a Eucalyptus selected from the species belonging to the sub-genera Eudesmia, Symphomyrtus and Corymbia and from the following species: Eucalyptus globulus L., Eucalyptus pulverulenta Sims, Eucalyptus kartzoffiana L. A. S. Johnson 1 Blaxell, Eucalyptus macrocarpa Hook., Eucalyptus cinerea F. Muell.ex Benth., Eucalyptus dorrigoensis (Blakely) L. A. S. Johnson 1 K. D. Hill, Eucalyptus leptopoda Benth., Eucalyptus occidentalis Endl., Eucalyptus viridis R. T. Baker, Eucalyptus polybractea R. T. Baker and Eucalyptus smithii R. T. Baker.

16. A compound of claim 15, wherein the Eucalyptus extract is selected from an extract of leaves, blossom, fruit, seeds, stems and trunk of Eucalyptus.

17. A pharmaceutical composition or food supplement comprising, as active ingredient, at least one compound of formula (I) of claim 13 in combination with an acceptable carrier.

18. The pharmaceutical composition or food supplement of claim 17, which comprises a Eucalyptus extract in which the proportion by weight of compound of formula (I) is greater than 0.05% and strictly less than 90%.

19. A method for the treatment and/or prevention of neurological or psychiatric ailments or pathologies and associated disorders, functional somatic disorders, obesity, overweight and dependence on addictive substances, ensuing from a dopamine and/or serotonin and/or noradrenaline reuptake disorder, in a subject in need thereof, comprising administration of an effective amount of at least one compound of claim 13 in the form of a medicament or food supplement.

20. The method of claim 19, wherein the treatment and/or the prevention of said neurological or psychiatric ailments or pathologies and associated disorders, functional somatic disorders, obesity, overweight and dependence on addictive substances, comprises an inhibition of the reuptake of dopamine and/or serotonin and/or noradrenaline.

21. The method of claim 19, wherein the neurological or psychiatric ailment or pathology or associated disorder, the functional somatic disorder, or the dependence on addictive substances is selected from the group comprising:
neurological diseases such as neurodegenerative diseases (Alzheimer's disease, Huntington's chorea, Parkinson's disease, cerebral vascular accidents, cranial traumatism), amyotrophic lateral sclerosis, senile dementias, fronto-temporal dementias, vascular dementias, migraine, neuropathic pains of central origin;
psychiatric diseases, such as depression (endogenous, resistant, reactive or iatrogenic), depressive state, schizophrenia, bipolar disorder, generalised anxiety, diseases associated with stress, panic attacks, obsessive compulsive disorders, post-traumatic stress syndromes, attention and hyperactivity disorders, eating behaviour disorders (especially bulimia, anorexia), phobia (especially agrophobia), autism;
memory, attention and vigilance disorders associated with neurological pathologies or psychiatric disorders;
functional somatic disorders such as chronic fatigue syndrome, fibromyalgia, irritable bowel syndrome, gastro-oesophageal refluxes, loss of libido, erection disorders, urinary incontinence;
dependence on addictive substances, especially nicotine, alcohol, opiates, cannabinoids, psychostimulants.

22. The method of claim 19, wherein the medicament is in oral, injectable or transdermal form.
